(19) United States
(12) Patent Application Publication
(10) Pub. No.: US 2005/0011527 A1
(43) Pub. Date: Jan. 20, 2005

(54) Hernia method with liquefied collagen

(76) Inventor: Allen B. Chefitz, New Rochelle, NY (US)

Correspondence Address: Allen B. Chefitz M.D.;Suite 301, 140 Lockwood Avenue, New Rochelle, NY, 10801, US

(21) Appl. No.: 10/784,473

(22) Filed: Feb. 23, 2004

Related U.S. Application Data
(60) Provisional application No. 60/448,709, filed on Feb. 21, 2003.

(51) Int. Cl. A61F 2/02

(52) U.S. Cl. 128/898; 623/23.72; 606/214

(57) ABSTRACT

A method for repair of a hernia whereby after dissection of a hernia, liquid or gel collagen is injected or sprayed into the resulting hernia space. The collagen then solidifies and serves as a barrier in place of a permanent prosthetic device. Properties of the collagen including viscosity may be modified through variation of temperature and pH.

### CROSS REFERENCE TO RELATED APPLICATION

This application claims priority to U.S. Provisional Application Ser. No. 60/448,709, which was filed on Feb. 21, 2003.

### BACKGROUND OF THE INVENTION

The most common method to repair a hernia involves implanting a synthetic, prosthetic material. One needs to deploy the device and secure it in place. Using such a permanent, prosthetic device carries risks, such as infection and pain, complications common to all foreign bodies. Intestinal submucosa of animals (Surgisis), a non-permanent material, has been used as a tissue graft and even as “fluidized graft compositions.” However, that material is non-human tissue and involves a more complex preparation. Alloderm (Lifecell) is derived from human tissue (skin), however, it is not employed as a liquid or injectable gel form. Using an injectable form of collagen, such as Alloderm, would simplify the minimally invasive techniques used to repair a hernia. Instead of placing a sheet of material through small trocar devices or ports, one would simply place a syringe with the contained fluid or gel-like collagen material into the hernia space and inject the material. The material would then solidify by adjusting the pH of the gel. The collagen matrix could be derived from human skin, further prepared with a described chemical and freeze drying process, and hydrated at certain temperatures to produce a fluid with varying viscosities depending on the temperature and pH of the solution.

### SUMMARY OF THE INVENTION

This invention involves repairing a hernia with an injectable, liquid collagen that becomes firmly adherent to its contained space. The solidified material would then serve as a barrier and, thus, repair the hernia.

### BRIEF DESCRIPTION OF THE DRAWING

FIG. 1 shows a partial sectional view of a hernia space injected with collagen.

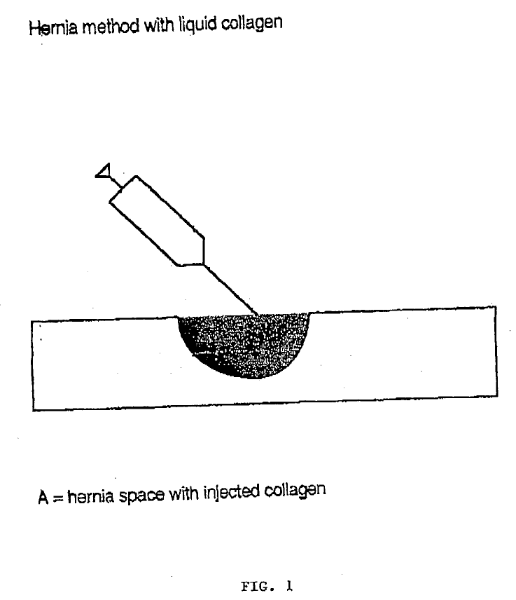

### DESCRIPTION OF THE INVENTION

This invention involves a method of repairing a hernia or body tissue defect. The surgeon dissects the hernia defect in the usual manner, performing a high dissection of the hernia space. Once the preperitoneal space is entered, the surgeon injects a liquid or gel collagen into the hernia space. Soon thereafter, the liquid collagen solidifies and repairs the hernia. Instead of using a mesh or other prosthetic device to repair the hernia, the surgeon simply injects or sprays a liquid that subsequently solidifies.

1. A method of repairing a hernia or body tissue defect comprising the injection of a liquid or gel collagen into a hernia space.
